(19) United States
(12) Patent Application Publication
(10) Pub. No.: US 2011/0313014 A1
(43) Pub. Date: Dec. 22, 2011

(54) PRODUCTS COMPRISING N-PHENYLPROPENOYL AMINO ACID AMIDES AND USES THEREOF

(75) Inventors: Fabien Robert, Divonne Les Bains (FR); Karin Kraehenbuehl, Fully (CH); Denis Marcel Barron, Lutry (CH); Jane Durga, Aigle (CH)

(73) Assignee: NESTEC S.A., Vevey (CH)

(21) Appl. No.: 13/148,516

(22) PCT Filed: Feb. 3, 2010
(86) PCT No.: PCT/EP2010/051263

§ 371 Date: Aug. 9, 2011

(30) Foreign Application Priority Data
Feb. 13, 2009 (EP) 09152775.4

(51) Int. Cl. A61K 31/405; A61P 25/28; A61P 25/24; A61P 25/20; A23G 1/32; A61P 3/10; A23L 1/305; A23J 1/00; A23F 5/00; A23G 1/56; A61P 25/00; A61P 27/02

(52) U.S. Cl. 514/419; 426/656; 426/425; 426/594; 426/593; 426/631; 426/2

(57) ABSTRACT

The present invention relates to use of a composition comprising N-phenylpropenoyl amino acid amides for the preparation of a product to treat or prevent neurodegenerative disorders, cognitive decline, mild cognitive impairment, dementia, mood disorders, depression, sleep disorders, diseases involving protein aggregation in a human or animal, Alzheimer's disease, macular degeneration or diabetes. The invention further relates to products comprising N-phenylpropenoyl amino acid amides, including food and beverage products, food supplements, and pet food products, and methods for affecting cognitive performance of humans and animals.

### FIELD OF THE INVENTION

The present invention relates to uses of N-phenylpropenoyl amino acid amides and to products comprising them, including food and beverage products, food supplements, and pet food products.

### BACKGROUND

Alzheimer's disease (AD) is a progressive neurodegenerative disease and the most common form of dementia, symptoms being e.g. memory loss, confusion, mood swings, and cognitive decline. It is characterized by the presence of extracellular amyloid plaques and intraneuronal neurofibrillary tangles in the brain, of which the main constituent is fibrillar aggregates of a 39-42 residue peptide referred to as the amyloid beta protein (Aβ). Aβfibril formation is thought to play a central role in the etiology of AD. Several pathogenic AD mutations have been shown to result in increased Aβ levels, especially of the variant Aβ42. Amyloid fibril formation is therefore thought to be the cause of disease progression and neurodegeneration in AD. It has been demonstrated by in vitro studies that Aβ fibril formation occurs via a complex multi-step mechanism that involves discrete soluble oligomeric intermediates termed ADDLS or protofibrils (PFs), which disappear upon fibril formation. This suggests that PFs may be AD's pathogenic species. A number of other diseases in humans and animals involve protein aggregation, e.g. macular degeneration, Bovine spongiform encephalopathy (BSE), Creutzfeldt-Jakob disease, and diabetes.

N-phenylpropenoyl amino acid amides have been found to occur naturally in coffee and cocoa, see e.g. Stark et al. 2006, J Agric Food Chem, 54, 2859-2867, as well as a number of other vegetable materials. WO 2008/009655 discloses that some N-phenylpropenoyl amino acid amides may be used for treatment of infections.

### SUMMARY OF THE INVENTION

The inventors have now found that N-phenylpropenoyl amino acid amides are effective to inhibit and/or retard the aggregation of amyloid beta peptides. Accordingly the invention relates to use of a composition comprising one or more N-phenylpropenoyl amino acid amides for the preparation of a product to treat or prevent neurodegenerative disorders, cognitive decline, mild cognitive impairment, dementia, mood disorders, depression, sleep disorders, diseases involving protein aggregation in a human or animal, Alzheimer's disease, macular degeneration or diabetes. In a further embodiment the invention relates to a food product, beverage product, food supplement or pet food product comprising N-phenylpropenoyl amino acid amide. In a still further embodiment the invention relates to non-therapeutical use of a food product, beverage product, food supplement or pet food product of the invention for treating and/or preventing cognitive decline, mood disorders, and/or sleep problems; for brain protection; and/or for improving cognitive performance, immune response, and/or gut barrier function, in a human or animal. And in another embodiment the invention relates to a method of improving cognitive performance; treating or preventing neurodegenerative disorders, cognitive decline, mild cognitive impairment, dementia, a disease involving protein aggregation, Alzheimer's disease, macular degeneration, or diabetes; the method comprising administering a food product, beverage product or pet food product comprising an effective amount of N-phenylpropenoyl amino acid amide, to a human or animal.

### BRIEF DESCRIPTION OF THE FIGURES

FIG. 1 shows the results of an assay of the ability of N-[(2E)-3-(3,4-dihydroxyphenyl)-1-oxo-2-propen-1-yl]-L-Tryptophan (CafTrp) (FIG. 1a) and N-[(2E)-3-(4-hydroxy-3-methoxyphenyl)-1-oxo-2-propen-1-yl]-L-Tryptophan (FerTrp) (FIG. 1b) to reduce and/or block the formation of amyloid fibrils from monomeric amyloid beta peptides.

FIG. 2 shows the results of an assay of the ability of CafTrp (FIG. 2a) and FerTrp (FIG. 2b) to reduce and/or block the formation of amyloid fibrils from protofibrils of amyloid beta peptides.

### DETAILED DESCRIPTION OF THE INVENTION

An N-phenylpropenoyl amino acid amide according to the present invention may be any N-phenylpropenoyl amino acid amide. Preferably, an N-phenylpropenoyl amino acid amide according to the invention is an N-phenylpropenoyl amino acid amide which is naturally present in vegetable material, e.g. an edible vegetable material, preferably material of the coffee plant and/or the cocoa plant. An N-phenylpropenoyl amino acid amide according to the invention may be an N-phenylpropenoyl L-amino acid amide or an N-phenylpropenoyl D-amino acid amide.

Preferably, an N-phenylpropenoyl amino acid amide according to the invention is a compound of the following structure:

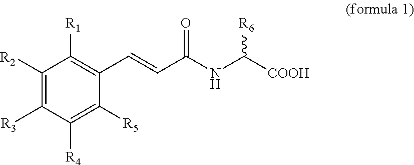

wherein a substituted cinnamic acid is linked by its carboxyl function to the amine group of an amino acid thereby forming an amide bond;
R6 is the side chain of the amino acid. If R6=H the amino acid would be glycine, if R6=methyl the amino acid would be alanine and so on. The preferred amino acids are: tyrosine, tryptophane, phenylalanine, histidine, aspartic acid and glutamic acid. The amino acids can be α-, β or γ amino acids, and α-amino acids can be in D or L configuration;

R1 to R5 is a substituent selected from hydroxy, methoxy (Me), hydrogen, or O-glycosyl. If R1 to R5 are all hydrogen, the phenylpropenoyl group is cinnamoyl. The preferred phenylpropenoyl groups are feruloyl (R2=OMe, R3=0H, R1=R4=R5=H), caffeoyl (R2=R3=OH, R1=R4=R5=H) and coumaroyl (R3=OH, R1=R2=R4=R5=H); and
the double bond can be in trans (E) or cis (Z) conformation.

More preferably, an N-phenylpropenoyl amino acid amide according to the invention is a compound selected from the group consisting of: N-[(2E)-3-(3,4-dihydroxyphenyl)-1-oxo-2-propen-1-yl]-L-Tryptophan; N-[(2E)-3-(4-hydroxyphenyl)-1-oxo-2-propen-1-yl]-L-Tryptophan; N-[(2E)-3-(3,4-dihydroxyphenyl)-1-oxo-2-propen-1-yl]-L-Tyro sine; N-[(2E)-3-(4-hydroxyphenyl)-1-oxo-2-propen-1-yl]-L-Tyrosine; N-[(2E)-3-(3,4-dihydroxyphenyl)-1-oxo-2-propen-1-yl]-L-Aspartic acid; N-[(2E)-3-(4-hydroxy-3-methoxyphenyl)-1-oxo-2-propen-1-yl]-L-Tryptophan; N-[(2E)-3-(3,4-dihydroxyphenyl)-1-oxo-2-propen-1-yl]-L-Phenylalanine; N-[(2E)-3-(4-hydroxyphenyl)-1-oxo-2-propen-1-yl]-L-Aspartic acid; N-[(2E)-3-(3,4-dihydroxyphenyl)-1-oxo-2-propen-1-yl]-3-hydroxy-L-Tyro sine; N-[(2E)-3-(4-hydroxy-3-methoxyphenyl)-1-oxo-2-propen-1-yl]-L-Aspartic acid; N-[(2E)-3-(4-hydroxyphenyl)-1-oxo-2-propen-1-yl]-3-hydroxy-L-Tyro sine; N-[(2E)-3-(3,4-dihydroxyphenyl)-1-oxo-2-propen-1-yl]-L-Glutamic acid; N-[(2E)-3-(4-hydroxyphenyl)-1-oxo-2-propen-1-yl]-L-Glutamic acid; N-[(2E)-3-phenyl-1-oxo-2-propen-1-yl]-L-Aspartic acid; N-[(2E)-3-(4-hydroxy-3-methoxyphenyl)-1-oxo-2-propen-1-yl]-L-Tyrosine.

In a preferred embodiment of the invention an N-phenylpropenoyl amino acid amide is obtained by extraction of a vegetable material. The vegetable material may be any vegetable material comprising one or more N-phenylpropenoyl amino acid amides, such as e.g. a coffee or cocoa material; material from Angelica archangelica, e.g. roots; material from Cassia angustifolica or Cassia senna, e.g. fruits; material from Coriandrum sativum, e.g. fruits; material from Hedera helix, e.g. leaves; material from Lavandula species, e.g. flowers; and/or material from Sambucus nigra, e.g. flowers.

Extraction of N-phenylpropenoyl amino acid amides from vegetable material may be performed by any suitable method known in the art. The extraction may be performed to achieve the necessary degree of purity for the specific product to be produced. For the production of a food product, a beverage product, a pet food product, or the like, by extraction of a food grade vegetable material, a high degree of purity may not always be needed. An extract comprising N-phenylpropenoyl amino acid amides, or enriched in N-phenylpropenoyl amino acid amides compared to the vegetable material from which it is extracted, may be sufficient. Extraction may e.g. be performed as liquid extraction used e.g. water, an alcohol, acetone, n-pentane, or any other suitable liquid, and/or mixes of such liquids, e.g. a mixture of water and an alcohol, e.g. ethanol, or a mixture of water and acetone; or solid phase extraction, e.g. using membranes or resins, e.g. a polymeric resin such as e.g. a polyvinylpolypyrrolidone (PVPP) resin. Suitable methods may be used to purify the extract to the required degree of purity, or enrichment, of the N-phenylpropenoyl amino acid amides, and/or to remove the extraction liquid. Pure N-phenylpropenoyl amino acid amides may not always be desired, it may be advantageous to use a combination of N-phenylpropenoyl amino acid amides, e.g. to increase the efficacy for the desired use, e.g. a combination of the specific N-phenylpropenoyl amino acid amides disclosed herein.

N-phenylpropenoyl amino acid amides may be synthesized, e.g. as described by Stark and Hofmann 2005, J. Agric. Food Chem., 53, 5419-5428; Stark et al. 2006, J. Agric. Food Chem., 54, 2859-2867; and Hensel et al. 2007, Planta Med, 73, 142-150; all of which are included herein by reference.

### Coffee

A coffee material according to the invention is any material derived from a coffee plant (i.e. a plant belonging to the genus Coffea, preferably Coffea arabica, Coffea canephora, or Coffea liberica), preferably coffee fruit (sometimes called coffee cherry) or coffee bean. The coffee material may be treated in any suitable way before it is extracted, it may e.g. be heat treated and/or roasted. Roasting of coffee beans is well known in the art of producing coffee products for consumption, and if coffee beans are used to extract N-phenylpropenoyl amino acid amides they may be roasted by conventional methods, or they may be green, un-roasted, coffee beans. Extraction of N-phenylpropenoyl amino acid amides from coffee material may preferably be done by liquid extraction using water; an alcohol, e.g. ethanol; or a mixture of water and alcohol, e.g. a water and ethanol mixture; or by solid phase extraction, e.g. using membranes or resins, e.g. a polymeric resin such as e.g. a polyvinylpolypyrrolidone (PVPP) resin. In one embodiment N-phenylpropenoyl amino acid amides are obtained by aqueous extraction of green and/or roasted coffee beans, methods for producing an aqueous extract of coffee beans are well known in the art, e.g. as practiced when brewing a cup of coffee, as well as in the art of soluble coffee production. Purification of N-phenylpropenoyl amino acid amides from an aqueous extract of coffee beans may e.g. be achieved by chromatography techniques, e.g. by Reversed Phase chromatography, or by solid phase extraction, e.g. using membranes or resins, e.g. a polymeric resin such as e.g. a polyvinylpolypyrrolidone (PVPP) resin

### Cocoa

A cocoa material according to the invention may be any material derived from a cocoa plant (Theobroma cacao), preferably from cocoa pod (the fruit of the cocoa plant), more preferably from cocoa bean. Cocoa material may be treated in any suitable way before extraction. Cocoa beans to be extracted may undergo fermentation and/or roasting before extraction. In a preferred embodiment of the invention cocoa material is cocoa powder and/or cocoa butter. Production of cocoa powder and cocoa butter is well known in the art, and is usually produced by removing pulp and beans from cocoa pods, fermenting the pulp and bean mixture, drying the fermented beans, roasting the beans, cracking the beans to produce cocoa nibs, and grinding the cocoa nibs to produce chocolate liquor, which may optionally be separated into cocoa powder and cocoa butter. Extraction of N-phenylpropenoyl amino acid amides from cocoa material may be performed by any suitable method, e.g. by liquid extraction using water; an alcohol, e.g. ethanol; or a mixture of water and alcohol, e.g. a water and ethanol mixture; or solid phase extraction, e.g. using membranes or resins, e.g. a polymeric resin such as e.g. a polyvinylpolypyrrolidone (PVPP) resin.

According to the present invention one or more N-phenylpropenoyl amino acid amides is/are used for preparation of a product, e.g. a food product, a beverage product, a food supplement, a pet food product or a medicament, to treat or prevent neurodegenerative disorders, cognitive decline, and/or diseases involving protein aggregation in a human or animal.

### Food and Beverage Products

A food product according to the invention may be any food product, such as e.g. a culinary product, e.g. a soup; a confectionery product, e.g. a chocolate product, such as a chocolate bar; a dairy product, e.g. a fermented milk product, such as a yogurt, or a cheese product; a cereal product; a bread product, e.g. a cake or biscuit; a condiment, e.g. mayonnaise or salad dressing; a meat product; an ice cream product; or the like. A beverage product may be any beverage product, such as e.g. a coffee beverage, e.g. black coffee, coffee with milk, cappuccino, a soluble coffee product, such as pure soluble coffee, or a coffee mix, e.g. comprising soluble coffee, creamer and/or whitener and/or sweetener; a tea beverage, e.g. ice tea; a milk beverage; a cocoa beverage; a malt beverage; a soft drink; mineral water, e.g. fortified and/or flavoured water; or the like. It is to be understood that a beverage product according to the invention may also be a preparation to be used for preparing a finished beverage, e.g. a powder or concentrate to which a liquid, e.g. water or milk, is to be added to prepare the finished beverage for consumption. In a preferred embodiment of the invention a beverage product is a coffee or cocoa beverage, or a beverage comprising a mixture of coffee and cocoa material. A food or beverage product according to the invention may be a medical food or beverage product aimed at fulfilling special nutritional needs of patients with a medical condition, weak or elderly persons, or other persons with specific nutritional needs.

A food, beverage, or pet food product of the invention comprises N-phenylpropenoyl amino acid amide, e.g. obtained by extraction from a vegetable material. In one embodiment the food, beverage, or pet food product consists of an extract of a N-phenylpropenoyl amino acid amide containing vegetable material, such as e.g. coffee beans or cocoa. In another embodiment N-phenylpropenoyl amino acid amide is added, e.g. as a synthesised compound or comprised in an extract of a vegetable material, at any appropriate step during the manufacture of the food, beverage, or pet food product. In a preferred embodiment N-phenylpropenoyl amino acid amide is comprised in an extract of coffee and/or cocoa used as an ingredient in the production of the food, beverage, or pet food product.

In one embodiment the invention relates to a food product, beverage product, food supplement or pet food product comprising at least 1000 mg of N-phenylpropenoyl amino acid amides per kg of dry matter, such as at least 2000 mg/kg dry matter, at least 5000 mg/kg dry matter, or at least 10.000 mg/kg dry matter. In a further embodiment the invention relates to a coffee product comprising at least 50 mg of N-phenylpropenoyl amino acid amides per kg of coffee solids, such as at least 200 mg/kg coffee solids, at least 500 mg/kg coffee solids, or at least 1000 mg/kg coffee solids. By the term coffee solids is meant all compounds and material originating from a coffee plant except for water. A coffee product according to the invention is a product based on coffee ingredients, e.g. roast and ground coffee; pure soluble coffee; a coffee mix, such as a mix of pure soluble coffee with creamer, whitener, sweetener, and or flavouring; a coffee beverage, e.g. black coffee, coffee with milk, cappuccino, café latte, or the like. In a preferred embodiment, at least 90%, such as at least 95%, at least 98% or at least 99%, of the N-phenylpropenoyl amino acid amide in the product is derived from coffee.

### Medicament

In one embodiment the invention relates to use of a composition comprising one or more N-phenylpropenoyl amino acid amides for the preparation of a medicament. The composition may further comprise any other suitable compound, e.g. a pharmaceutically acceptable carrier, adjuvant and/or salt. A pharmaceutically acceptable carrier or adjuvant includes any solvent, dispersion media, antibacterial or antifungal agent and the like, that are physiologically acceptable and suitable for the desired route of administration. The medicament may e.g. be suitable for oral, intravenous, intramuscular or subcutaneous administration.

### Use and Method

The invention relates to use of a composition comprising N-phenylpropenoyl amino acid amides for the preparation of a product to treat or prevent neurodegenerative disorders, cognitive decline, mild cognitive impairment, dementia, mood disorders, depression, sleep disorders, a disease involving protein aggregation, Alzheimer's disease (including common symptoms of AD, dementia, mild cognitive impairment and cognitive decline like sleep disorders, mood swings, depression, stress), macular degeneration, or diabetes. The invention further relates to non-therapeutical use of a food product, beverage product, food supplement or pet food product of the invention, for treating and/or preventing cognitive decline, mood disorders, and/or sleep problems; for brain protection; and/or for improving cognitive performance, immune response, and/or gut barrier function in a human or animal. Cognitive performance may e.g. be expressed as ability and speed of learning, ability and speed of solving intellectual problems, ability to form and recall memories, reaction time, and the like. Cognitive decline may e.g. manifest itself as reduced memory, forgetfulness, word or name-finding problems, decline in memory, concentration, ability to plan or organise, ability to perform complex tasks, and/or cognitive performance, and may e.g. result from age, stress, disease, or other grounds. Cognition is understood as mental processes such as comprehension, inference, decision-making, planning, learning, memory, association, concept formation, language, attention, perception, action, problem solving and mental images.

In a further embodiment, the invention relates to a method of improving cognitive performance; treating or preventing neurodegenerative disorders, cognitive decline, mild cognitive impairment, dementia, a disease involving protein aggregation, Alzheimer's disease, macular degeneration or diabetes; the method comprising administering a food product, beverage product or pet food product comprising an effective amount of N-phenylpropenoyl amino acid amide, to a human or animal. In a still further embodiment, the invention relates to a method of treating or preventing neurodegenerative disorders, cognitive decline, mild cognitive impairment, dementia, a disease involving protein aggregation, Alzheimer's disease, macular degeneration or diabetes, comprising administering an effective amount of a medicament comprising a N-phenylpropenoyl amino acid amide to a human or animal in need thereof. The food product, beverage product or pet food product may be administered concomitantly with a medicament to increase the efficacy and/or reduce the dose of the medicament.

The uses and methods of the invention may be performed in such a way as is most suitable to achieve the desired result. For example, if the product is a food product, beverage product or pet food product, the amount of N-phenylpropenoyl amino acid amides comprised in the product may be such as to achieve the desired effect in an individual consuming a single serving e.g. once a week, every second day, once a day, or 2-4 times a day, without resulting in undesired effects, e.g. on taste or appearance of the product. If the product is a food supplement or medicament, the amount of N-phenylpropenoyl amino acid amides comprised in the product and the frequency of administration may be such as to maximise the desired effect while minimising adverse effects, if any.

If the product is a food product, beverage product or pet food product, one serving of the product may e.g. comprise at least 100 microgram N-phenylpropenoyl amino acid amides, such as 500 microgram, 1000 microgram or 5000 microgram N-phenylpropenoyl amino acid amides. In one embodiment, the product comprises at least 1000 mg of N-phenylpropenoyl amino acid amides per kg of dry matter, such as at least 2000 mg/kg dry matter, at least 5000 mg/kg dry matter, or at least 10.000 mg/kg dry matter.

### EXAMPLES

### Example 1

N-[(2E)-3-(3,4-dihydroxyphenyl)-1-oxo-2-propen-1-yl]-L-Tryptophan (CafTrp) and N-[(2E)-3-(4-hydroxy-3-methoxyphenyl)-1-oxo-2-propen-1-yl]-L-Tryptophan (FerTrp) were synthesized and the effect of these compounds on the formation of amyloid beta aggregates were tested in the following assays:

### Monomeric Aβ

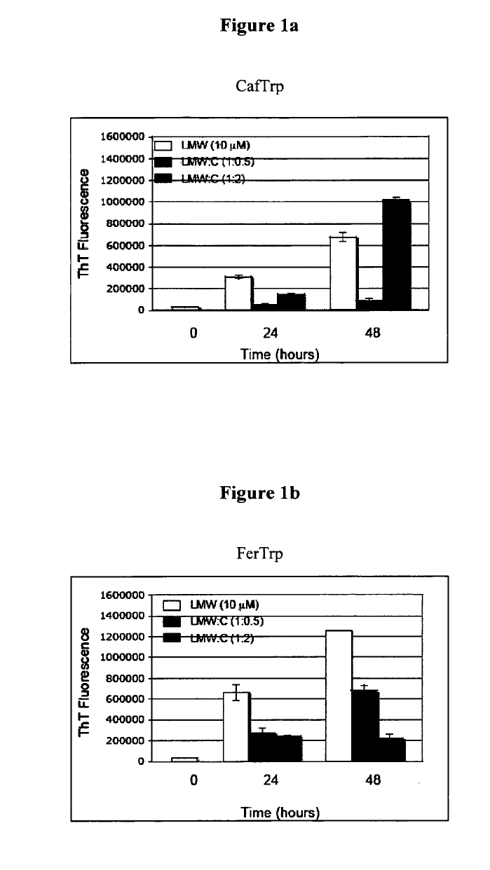

Monomeric Aβ42 peptides were purified by size exclusion chromatography and incubated at 37° C. at a concentration of 10 μM with the compound of interest at a ratio of Aβ42 to the tested compound of 1:0.5 and 1:2 (molar ratio). The extent of aggregation was assessed at 24 and 48 hours by Thioflavin T (ThT) fluorescence. Controls were performed in the same way except for the absence of a compound to be tested. ThT is a hydrophobic dye that exhibits enhanced fluorescence upon binding to amyloid fibrils. ThT binds specifically to amyloid fibrils, but not monomeric forms of A13. In this assay a decrease or absence of ThT fluorescence indicated that the molecule being tested reduced and/or blocked the formation of amyloid fibrils. The results of this assay are shown in FIG. 1 (FIG. 1a: CafTrp; FIG. 1b: FerTrp).

### Protofibrillar Aβ

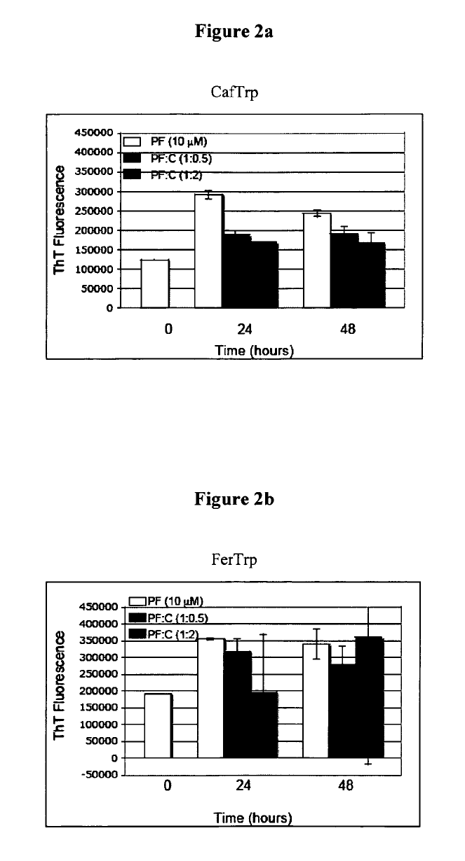

Size exclusion purified protofibrillar mixture of Aβ42 was incubated at 37° C. at a concentration of 10 μM with the compound of interest at a ratio of Aβ42 to the tested compound of 1:0.5 and 1:2 (molar ratio). The extent of aggregation was assessed at 24 and 48 hours by Thioflavin T (ThT) fluorescence. Controls were performed in the same way except for the absence of a compound to be tested. A decrease or absence of an increase in ThT fluorescence signal of protofibrils indicated that the molecule being tested reduced and/or blocked the formation of amyloid fibrils. The results of this assay are shown in FIG. 2 (FIG. 2a: CafTrp; FIG. 2b: FerTrp).

1. A product comprising at least 1000 mg of N-phenylpropenoyl amino acid amide per kg of dry matter.

2. A product according to claim 1, wherein the N-phenylpropenoyl amino acid amide is obtained by extraction of a coffee and/or cocoa material.

3. A product according to claim 1, wherein the N-phenylpropenoyl amino acid amide is selected from the group of compounds consisting of N-[(2E)-3-(3,4-dihydroxyphenyl)-1-oxo-2-propen-1-yl]-L-Tryptophan; N-[(2E)-3-(4-hydroxyphenyl)-1-oxo-2-propen-1-yl]-L-Tryptophan; N-[(2E)-3-(3,4-dihydroxyphenyl)-1-oxo-2-propen-1-3/1]-L-Tyro sine; N-[(2E)-3-(4-hydroxyphenyl)-1-oxo-2-propen-1-yl]-L-Tyrosine; N-[(2E)-3-(3,4-dihydroxyphenyl)-1-oxo-2-propen-1-yl]-L-Aspartic acid; N[(2E)-3-(4-hydroxy-3-methoxyphenyl)-1-oxo-2-propen-1-yl]-L-Tryptophan; N[(2E)-3-(3,4-dihydroxyphenyl)-1-oxo-2-propen-1-yl]-L-Phenylalanine; N-[(2E)-3-(4-hydroxyphenyl)-1-oxo-2-propen-1-yl]-L-Aspartic acid; N-[(2E)-3-(3,4-dihydroxyphenyl)-1-oxo-2-propen-1-yl]-3-hydroxy-L-Tyrosine; N-[(2E)-3-(4-hydroxy-3-methoxyphenyl)-1-oxo-2-propen-1-3/1]-L-Aspartic acid; N-[(2E)-3-(4-hydroxyphenyl)-1-oxo-2-propen-1-yl]-3-hydroxy-L-Tyrosine; N-[(2E)-3-(3,4-dihydroxyphenyl)-1-oxo-2-propen-1-yl]-L-Glutamic acid; N-[(2E)-3-(4-hydroxyphenyl)-1-oxo-2-propen-1-yl]-L-Glutamic acid; N-[(2E)-3-phenyl-1-oxo-2-propen-1-yl]-L-Aspartic acid; and N-[(2E)-3-(4-hydroxy-3-methoxyphenyl)-1-oxo-2-propen-1-yl]-L-Tyrosine.

4. A product according to claim 1 being wherein the product is selected from the group consisting of a coffee beverage, or cocoa beverage, and a chocolate product.

5. A coffee product comprising at least 50 mg of N-phenylpropenoyl amino acid amide per kg of coffee solids.

6. A method for treating or preventing a disorder selected from the group consisting of neurodegenerative disorders, cognitive decline, mild cognitive impairment, dementia, mood disorders, depression, sleep disorders, diseases involving protein aggregation in a human or animal, Alzheimer's disease, macular degeneration and diabetes comprising administering a composition comprising N-phenylpropenoyl amino acid amides to a mammal in need of same.

7. Method according to claim 6 wherein the N-phenylpropenoyl amino acid amides is/are obtained by extraction of a vegetable material.

8. Method according to claim 7 wherein the N-phenylpropenoyl amino acid amides is/are obtained by extraction of a coffee and/or cocoa material.

9. Method according to claim 8 wherein the coffee and/or cocoa material is heat treated and/or roasted before extraction of N-phenylpropenoyl amino acid amide.

10. Method according to claim 6 wherein the N-phenylpropenoyl amino acid amides is selected from the group of compounds consisting of N-[(2E)-3-(3,4-dihydroxyphenyl)-1-oxo-2-propen-1-yl]-L-Tryptophan; N-[(2E)-3-(4-hydroxyphenyl)-1-oxo-2-propen-1-yl]-L-Tryptophan; N-[(2E)-3-(3,4-dihydroxyphenyl)-1-oxo-2-propen-1-yl]-L-Tyro sine; N-[(2E)-3-(4-hydroxyphenyl)-1-oxo-2-propen-1-yl]-L-Tyrosine; N-[(2E)-3-(3,4-dihydroxyphenyl)-1-oxo-2-propen-1-yl]-L-Aspartic acid; N-[(2E)-3-(4-hydroxy-3-methoxyphenyl)-1-oxo-2-propen-1-yl]-L-Tryptophan; N-[(2E)-3-(3,4-dihydroxyphenyl)-1-oxo-2-propen-1-yl]-L-Phenylalanine; N-[(2E)-3-(4-hydroxyphenyl)-1-oxo-2-propen-1-yl]-L-Aspartic acid; N-[(2E)-3-(3,4-dihydroxyphenyl)-1-oxo-2-propen-1-yl]-3-hydroxy-L-Tyrosine; N-[(2E)-3-(4-hydroxy-3-methoxyphenyl)-1-oxo-2-propen-1-yl]-L-Aspartic acid; N-[(2E)-3-(4-hydroxyphenyl)-1-oxo-2-propen-1-yl]-3-hydroxy-L-Tyrosine; N-[(2E)-3-(3,4-dihydroxyphenyl)-1-oxo-2-propen-1-yl]-L-Glutamic acid; N-[(2E)-3-(4-hydroxyphenyl)-1-oxo-2-propen-1-yl]-L-Glutamic acid; N-[(2E)-3-phenyl-1-oxo-2-propen-1-yl]-L-Aspartic acid; and N-[(2E)-3-(4-hydroxy-3-methoxyphenyl)-1-oxo-2-propen-1-yl]-L-Tyrosine.

11. Method according to claim 6 wherein the composition is selected from the group consisting of a food product, a beverage product, a food supplement, a pet food product and a medicament.

12. Method according to claim 6 wherein the composition is selected from the group consisting of a coffee beverage, cocoa beverage, and or a chocolate product.

13. A non-therapeutical method for treating and/or preventing cognitive decline, mood disorders, sleep problems, for brain protection; for improving cognitive performance, immune response, and gut barrier function, in a human or animal comprising administering a composition a product comprising at least 1000 mg of N-phenylpropenoyl amino acid amide per kg of dry matter.

14. A method of improving cognitive performance treating or preventing neurodegenerative disorders, cognitive decline, mild cognitive impairment, dementia, a disease involving protein aggregation, Alzheimer's disease, macular degeneration, and diabetes, the method comprising administering a product comprising an effective amount of N-phenylpropenoyl amino acid amide, to a human or animal.
